(19) United States
(12) Patent Application Publication
(10) Pub. No.: US 2026/0315094 A1
(43) Pub. Date: Oct. 15, 2026

(54) CATHETER HAVING DIRECTIONAL TRANSDUCER

(71) Applicants: Hue-Teh Shih, Houston, TX (US); Jian-Chiun Liou, Hsinchu City (TW); Chen-Chia Chou, New Taipei City (TW)

(72) Inventors: Hue-Teh Shih, Houston, TX (US); Jian-Chiun Liou, Hsinchu City (TW); Chen-Chia Chou, New Taipei City (TW)

(21) Appl. No.: 19/467,517

(22) Filed: Feb. 2, 2026

Related U.S. Application Data
(63) Continuation of application No. 17/235,500, filed on Apr. 20, 2021, now abandoned.

(51) Int. Cl. A61B 17/32; A61B 17/00

(52) U.S. Cl. CPC A61B 17/320068; A61B 17/320016; A61B 2017/00039; A61B 2017/00044; A61B 2017/00867; A61B 2017/320069

(57) ABSTRACT

Systems and methods for noncontact ablation of tissues or materials in/on the body using an ablation catheter that has a directional ablation unit. The ablation unit has a set of individual transducer (e.g., ultrasound) elements that are movably positioned. A position adjustment element (a deformer) is movable with respect to the ultrasonic transducer to alter the positions of the individual transducer elements and thereby alter the directions in which the transducer elements emit ultrasonic energy. In one embodiment, the ablation catheter is a lumen catheter through which a positioning/measurement catheter can be inserted. The luminal ablation catheter is inserted through the skin and into the body via conventional methods. The ablation and positioning catheters can be used to make electrical measurements with respect to the surrounding tissue, and the ablation catheter can be used to emit energy in a desired direction and/or pattern to ablate target tissue.

### CROSS-REFERENCE TO RELATED APPLICATIONS

This application is a continuation application that is related to, and claims priority to, the co-pending U.S. patent application having the Ser. No. 17/235,500, filed on Apr. 20, 2021, having the title of “Catheter Having Directional Transducer.” The above-referenced patent application is incorporated by reference herein in its entirety.

### FIELD OF THE INVENTION

The invention relates generally to medical devices and methods for their use. More particularly, the invention relates to systems and methods for imaging and ablating tissues or materials within the body using a directional transducer.

### BACKGROUND

In the medical field, ablation is used as a therapy in the treatment of various diseases (e.g., skin spots, snoring, tumors, hemorrhage, arrhythmia, atherosclerosis) and has a number of modalities (e.g., DC, radio frequency (RF), microwave, laser, ultrasound, chemical, cryogenic, rotary blade).

An ablation can be performed to transect or otherwise alter the function of some tissue. For example, fatty deposits or plaque which present an obstruction in an artery can be removed using a laser to break up the obstruction, thereby restoring blood flow.

In another example, cardiac muscle can be burned to create a lesion which obstructs the conduction of (aberrant) electrical signals within the heart (that is, the lesion interrupts abnormal electrical conduction in the heart, therefore cures abnormal heart rhythms.)

The treatment of cardiac arrhythmia is of particular interest, as it is debilitating or even deadly and is one of the most common disorders in clinical practice. In the United States, many patients die every year due to specific heart rhythm disorders that are caused by abnormal, rapid beats. For example, it is estimated that 3 million people in the United States have atrial fibrillation, one of the most common causes of stroke. Additionally, atrial fibrillation is also related to heart failure. A number of pharmacological and surgical therapies are available to treat these disorders.

Percutaneous transluminal (catheter-based) ablation is a minimally invasive therapy and has been shown to be relatively safe and effective in treating selected heart rhythm disorders. In a typical cardiac ablation procedure, several catheters are advanced through the venous or arterial systems and positioned inside the heart. These are used to assess the etiology of the disease and then to treat it.

Various procedures are employed in catheter-based ablation therapy. Typically, a single percutaneous procedure to ablate cardiac tissue is iterative and makes use of multiple sheaths and catheters in multiple steps. First, catheters are maneuvered into various positions to denote the location and measure the timing of cardiac activation. This is followed by the placement of an ablation catheter in contact with the cardiac tissue at a location where electrical activity is to be disrupted. The ablation catheter is used to burn or freeze the engaged tissue, altering the tissue behavior. Additional measurements are then made to reassess the cardiac function. This process is performed iteratively, alternating measurement and ablation, until the cardiac activation and resulting heart rhythm are modified as desired.

Multiple factors affect the success of an ablation procedure. For example, because it is essential to have an accurate diagnosis of the type of arrhythmia and localization of the culprit(s) of the arrhythmia to achieve success in the ablation procedure, it is often necessary to place multiple electrode catheters in the heart. These catheters can interfere with one another as they are manipulated, and the use of multiple catheters can also increase the risks of clot formation and injury to the cardiac tissue.

Another factor is contact with the tissue to be ablated. Conventional devices such as those that use radio frequency energy, cryogenic material, and laser energy to ablate tissue are designed to ablate the target tissue when the catheter is in direct contact with the tissue. Tissue contact is critical, but various factors affect the contact and contact pressure between the tissue and ablation element, including the anatomy of the target and the adjacent structure, the design of the catheter itself, and the relationship between the catheter and the anatomy, to list only a few. Additionally, the presence of blood between the tissue and the ablation element severely decreases the efficacy of radio frequency, cryogenic, and laser ablation. The tissue characteristics can also affect the efficacy of the ablation energy. For instance, it is difficult for radio frequency and cryogenic materials to penetrate the superficial scar tissue and reach the deeper muscular tissue of interest.

Another factor affecting the success of an ablation procedure is the positioning of the catheters. The positioning not only affects the ability to take consistent measurements with a recording electrode, but also affects the ability to reliably ablate the intended target tissue. For instance, mispositioning of the ablating element/electrode can result in failure to return to an ablation site to complete a burn (an ablation), or can result in gaps in a line of burns when creating a linear lesion. These factors can make it difficult to apply the therapy, render the therapy ineffective, or even enhance the disease (e.g., make the cardiac tissue proarrhythmic).

It would therefore be desirable to provide systems and methods for ablating tissue in the body which overcome one or more of the problems associated with prior art ablation systems and methods.

### SUMMARY OF THE INVENTION

One or more of the problems outlined above may be solved by the various embodiments of the invention. Broadly speaking, the invention includes systems and methods for ablating body tissues using catheters that have directional transducers.

One embodiment comprises a system for noncontact ablation of tissues or materials in or on the body using a sheath-like luminal catheter and a second catheter that is inserted into the lumen of the first catheter. In one embodiment, the luminal catheter is an ablation catheter that also serves as the sheath for the other catheter, which is a positioning catheter. The luminal catheter incorporates a noncontact ablation unit such as an ultrasonic transducer unit, and may also include recording electrodes. The ultrasonic transducer unit itself has a set of individual transducer elements that are movably positioned. A position adjustment element (a deformer) is movable with respect to the ultrasonic transducer to alter the positions of the individual transducer elements and thereby alter the directions in which the transducer elements emit ultrasonic energy. The luminal catheter is inserted through the skin and into the body (e.g., into the heart, tubular structures such as veins, or other organs or tissues) via conventional methods. The positioning catheter can be inserted into the lumen so that the ablation catheter and the positioning catheter are simultaneously positioned to record the electrograms and ablate the target tissue. The position adjustment element can then be moved to adjust the positions of the transducer elements so that the ablation energy will be directed in a desired direction or pattern toward the target tissue.

In another embodiment, instead of a mechanical deformer, an electronic device can be used to alter the alignment of the transducers to change the direction of the ablation force. For example, shape-memory materials can be used for portions of the transducer structure, and various levels of electric current and heat can be applied to these materials to cause them to be deformed (i.e., to assume specific shapes at the different levels of electric current and heat).

Either or both of the ablation and positioning catheters can be steerable, and either or both may incorporate recording electrodes. The positioning catheter may be configured with a loop or other shape to aid in maintaining proper positioning in the body. The loop may be off-axis with respect to the main body of the system in order to allow adjustment of the distance between the ablation unit and the target tissue. The noncontact ablation unit of the ablation catheter may, for example, have an array of cylindrically arranged transducer elements which radiate energy in a circumferential or semi-circumferential pattern. The movable elements of the ablation unit allow the adjustment of the direction or pattern in which the energy is radiated. Specific ones of the ablation elements may also be individually controlled to affect the pattern in which the energy is radiated. In other words, with multiple transducers forming multiple ablation elements, different patterns of ablation points, lines, or areas can be achieved by applying ablation energy through designated corresponding ablation elements. The ablation catheter is particularly well-suited for applications in which the target is shaped in a tubular or funnel-like fashion, but is not limited to such structures.

An alternative embodiment comprises a method for noncontact ablation of target tissue that includes providing a first catheter which has a lumen and a second catheter configured to fit within the lumen of the first catheter. One of the first and second catheters is a noncontact ablation catheter and one is a positioning catheter. The method further includes inserting the first catheter into a body and placing the second catheter within the lumen of the first catheter. The first catheter is positioned with respect to the target tissue using the second catheter, and the positions of the ablation elements on the first catheter are moved to adjust the pattern and/or direction in which the ablation energy is emitted from the catheter to the target tissue. One or more recordings may be made using the catheters, and target tissue in the body is ablated using the ablation catheter. The recordings are typically, but not limited to, electrograms.

Numerous additional embodiments are also possible.

### BRIEF DESCRIPTION OF THE DRAWINGS

Other objects and advantages of the invention may become apparent upon reading the following detailed description and upon reference to the accompanying drawings.

FIG. 1 is a diagram illustrating catheter placement within a body for a cardiac ablation procedure in accordance with the prior art.

FIGS. 2A and 2B are diagrams illustrating the creation of lesions in cardiac tissue using conventional ablation techniques.

FIG. 3 is a diagram illustrating an exemplary embodiment of the invention.

FIGS. 4A-4D are diagrams illustrating principles of operation of directional non-contact ablation units in accordance with some embodiments.

FIGS. 5A and 5B are a pair of diagrams illustrating an exemplary directional non-contact ablation unit in accordance with some embodiments.

FIGS. 6A-6C are diagrams illustrating an ablation catheter including the directional non-contact ablation unit of FIGS. 5A and 5B.

FIGS. 7A and 7B are a pair of diagrams illustrating subsets of the catheter components in accordance with the embodiment of FIGS. 6A-6C.

FIGS. 8A and 8B are diagrams illustrating one embodiment of the present system positioned for an ablation procedure in a pulmonary vein.

FIG. 9 is a diagram illustrating an electronic controller that is coupled to the catheter system in accordance with some embodiments.

FIG. 10 is a diagram illustrating the independent controllability of individual ablation elements in accordance with some embodiments.

FIGS. 11A and 11B are diagrams illustrating the use of a single column of elements in the ablation unit to generate a linear lesion in accordance with some embodiments.

FIGS. 12A and 12B are diagrams illustrating the use of a single circumferential row of elements in the ablation unit to generate a thin circumferential lesion in accordance with some embodiments.

FIGS. 13A and 13B are diagrams illustrating the use of a selected set of elements in the ablation unit to generate a curved lesion in accordance with some embodiments.

While the invention is subject to various modifications and alternative forms, specific embodiments thereof are shown by way of example in the drawings and the accompanying detailed description. It should be understood that the drawings and detailed description are not intended to limit the invention to the particular embodiments which are described. This disclosure is instead intended to cover all modifications, equivalents and alternatives falling within the scope of the present invention as defined by the appended claims.

### DETAILED DESCRIPTION OF EXEMPLARY EMBODIMENTS

One or more of the problems outlined above may be solved by the various embodiments of the invention. It should be noted that the embodiments described below are exemplary and are intended to be illustrative of the invention rather than limiting.

Broadly speaking, the invention includes systems and methods for ablation of tissue in the body using directional ablation elements. In the medical field, many ablation devices and procedures have been developed to treat a variety of conditions. In particular, catheter-based devices have been developed to deliver therapies within various structures and spaces within the body. For example, the devices can be used for recanalizing atherosclerotic vessels, elimination of endometriosis on a uterine wall, disruption of conduction paths associated with reentrant tachycardia, and even the occlusion of blood vessels.

One embodiment consists of a combination of positioning and ablation catheters. The catheters may also perform measuring and recording functions. The ablation catheter is a sheath-like catheter that has an ultrasonic transducer affixed to the distal end. The recording/ablation catheter is configured to accept a positioning catheter through its lumen. The positioning catheter may serve to guide and anchor the ablation/recording catheter so that it can be accurately positioned. The positioning catheter may also include electrodes for recording tissue data. The ablation catheter is free to move along the recording/positioning catheter and so that it stands off from the tissue to be ablated (being supported by the positioning catheter). When deployed, the positioning catheter extends beyond the distal tip of the ablation catheter and engages some structure beyond the ablation catheter. The ultrasonic transducer is configured to emit energy to the target tissue. The electrodes on the ablation and positioning catheters are configured to record signals that are used to determine the mechanism of arrhythmia, the target of ablation, and the efficacy of an ablation. Recordings made during the ablation may be used as feedback to control the ablation.

Before describing the invention in detail, it will be helpful to understand how certain terms are used in the present disclosure. Very simplified descriptions of both normal and abnormal conduction in the heart will also be presented to aid in the understanding of cardiac tissue ablation and related devices and procedures.

“Tissue,” as used herein, refers to any material in the body, so tissue which is the target of ablation should not be narrowly construed to include only biological tissue, but may include targeted material, whatever its makeup or origin (e.g., muscle, tumor, plaque).

“Catheter” refers to a rod- or tube-like device inserted into the body. Many catheters, such as those used in cardiac ablations, are very narrow and elongated (similar to a wire) so that they can be inserted through the skin and into a blood vessel. This allows the catheters to provide access to, or to deliver some distal end-effector (therapy), to some site in the body. Catheters may have many different configurations (e.g., lumen, non-lumen, etc.) and many different functions (e.g., positioning, recording, ablation, etc.)

“Distal” refers to a point or end of an object which is opposite a reference point on the object. In regard to a catheter, the reference point is typically the end of the catheter external to the body, so the distal end of the catheter is the end which is inserted into the body.

“Proximal” refers to a point or end of an object which is nearest the reference point on the object. In reference to a catheter, the proximal end of the catheter is the end which is external to the body and is typically in the hands of the clinician.

A “lumen” is a passage or duct. A lumen in a catheter or sheath is a duct through the catheter. For the purposes of this disclosure, a “lumen catheter” or “luminal catheter” is a sheath-like catheter or hybrid catheter-sheath which has a lumen inside the catheter through which another catheter can be inserted into the body.

“Contact” refers to the physical contact between items such as a catheter and targeted tissue. For proper operation, a contact ablation device must directly engage the targeted tissue.

“Non-contact” refers to the lack of physical contact between items. For example, a “non-contact” ablation device is designed to stand off from the targeted tissue and ablate the tissue without directly contacting the tissue.

Conduction in the heart will now be briefly described.

The heart is constructed of muscles (myocardium), valves, and a control system. The myocardium not only provides the force behind the pumping action but supports the conduction of electrical signals. The heart also has a specialized conduction system whose purpose is to initiate and coordinate the pumping action of the muscle cells. One part of this system is the sinoatrial (SA) node. The SA node is the pacemaker of the heart and is responsible for initiating the heartbeat. The atria first receive the signal from the SA node, pumping blood into the ventricles. Simultaneously, the signal travels to the atrioventricular (AV) node. The signal then travels into the lower ventricles by means of Purkinje fibers causing the ventricles to contract “upwards” towards the valves.

For a given myocardial cell, there is a cycle of activation, recovery and waiting. Typically, a cell has an external stimulus such as from a neighboring cell (though a cell can spontaneously depolarize, which is referred to as “automaticity”). When activated, the cell “depolarizes” causing it to contract, and then “repolarizes”. The cell's activity stimulates neighboring cells (as will those cells) resulting in a “wave” of activity through the myocardium (a.k.a., activation front). There is a “refractory” period during this cycle when the cell cannot be stimulated. If a cell is ablated, it becomes nonfunctional and can no longer be stimulated, nor can it conduct a wave/signal to its neighbors.

There are a number of conditions that result in the disruption of the normal cardiac rhythm. One of these is sick sinus syndrome, which is the result of erratic SA node activity. Alternatively, the SA node activity can be inappropriately fast, causing inappropriate sinus tachycardia. In a case of inappropriate sinus tachycardia, the SA node can be ablated (burned or frozen), destroying the nodal tissue and thereby slowing down the rate of its firing to control the tachycardia.

Another disruption of normal cardiac rhythm is caused by the aberrant behavior of a cardiac cell or small number of cardiac cells that take over the pacing of the heart from the SA node. These cells prematurely depolarize, thereby initiating the depolarization of neighboring cells and resulting in an unwanted wave of electrical activity. This unwanted origin of electrical activity (a.k.a., ectopic focus) can be eliminated by destroying the offending cells with a localized burn (or freeze), allowing the SA node to again properly pace the heart.

Yet another arrhythmia is caused by the conduction of signals from an atrium directly to a ventricle (outside the specialized conduction system) via an (unwanted) accessory pathway. This pathway can be obstructed by burning (or freezing) the associated tissue.

Another form of arrhythmia is reentrant tachycardia, which is the result of a circuit incorporating tissue that effectively causes a signal delay. The delayed signal then initiates activity in adjacent tissue. The delayed signal re-enters the tissue, resulting in a self-perpetuating electrical circuit.

These are but a few simple examples. In each case, an ablation can be used to “correct” the abnormality. However, to do so requires the careful use of an ablation system to properly place an appropriately sized lesion.

As noted above, the treatment of cardiac arrhythmia is of particular interest since arrhythmia is one of the most common disorders in clinical practice and leads to so many fatalities. While a number of pharmacological and surgical therapies are available to treat these heart rhythm disorders, catheter ablation often proves to be curative.

Percutaneous transluminal catheter ablation is a minimally invasive therapy in which a series of catheters are inserted through the skin and advanced through the venous or arterial systems and positioned inside the heart or other tissues to either assess the etiology of or to treat the disease. Typically, a catheter or series of catheters are used to record the electrical signals from the location of interest and measure timing of cardiac activation followed by placement of an ablation catheter. The ablation catheter is then used to burn (or freeze) the engaged tissue, altering the tissue behavior. This process is typically performed iteratively, thereby modifying the heart rhythm. Percutaneous transluminal catheter ablation has been shown to be relatively safe and effective, for example, in treating selected heart rhythm disorders.

Ablation procedures typically employ a combination of devices appropriate to the task. Many of the devices are not directly involved in the ablation of the target tissue. These may include, for instance, recording/measurement catheters that aid in determining performance of an organ/structure, determining the etiology of a disease, or in evaluating the efficacy of an ablation. Positioning/anchoring catheters/sheaths may also be used to guide placement of the ablation and recording/measurement catheters to the desired locations in the heart or other tissues.

In the past, direct current (DC) ablation was used for the treatment of heart rhythm disorders, but radiofrequency (RF) ablation has supplanted it as the therapy of choice. For example, RF catheter ablation has revolutionized the treatment of supraventricular tachycardias (i.e., inappropriate sinus tachycardia, Wolff-Parkinson-White syndrome, atrioventricular nodal reentrant tachycardia, atrial tachycardia, and atrial flutter.)

Generally, a cardiac ablation entails directing a sufficient amount of energy into the tissue through a catheter in such a way as to produce a lesion within the heart tissue. Commonly, radiofrequency (RF) energy is used. The energy is directed into the myocardium (cardiac muscle) at a spot on the endocardium for a period great enough to produce a lesion sufficiently large to either destroy an ectopic focus or to obstruct an unwanted pathway. The RF ablation requires that the active electrode of the catheter be placed in continuous, direct contact with the target tissue for the prescribed period. Afterwards, recordings and measurements are made to determine if the lesion has the desired effect. If not, the ablation catheter may need to be (re)placed at the same ablation site to enlarge the lesion or placed at another (usually adjoining) site to create an additional (larger) lesion. This process continues until the procedure is successful or until the procedure is terminated for other reasons.

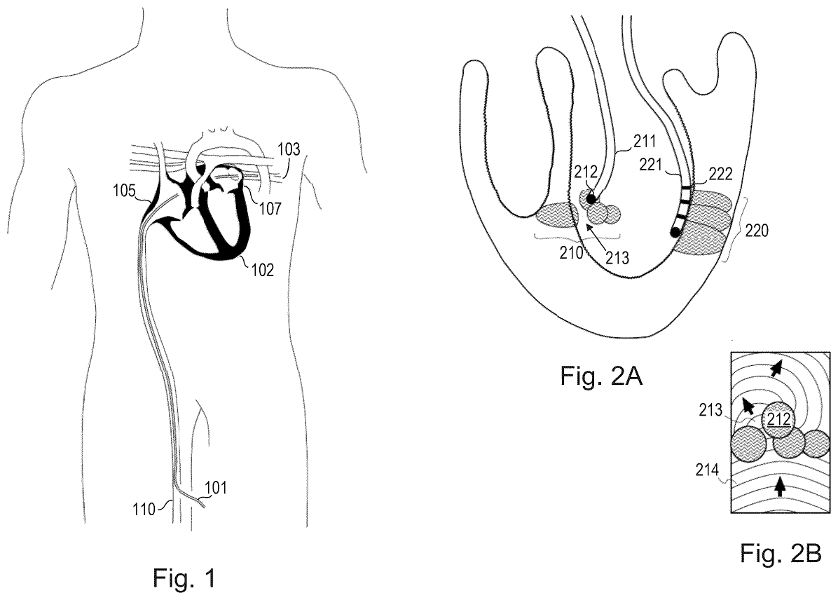

FIG. 1 illustrates a conventional configuration for ablation within a heart. Access to the heart (102) is provided through the circulatory system itself, typically a femoral vein (110). Other vessels (e.g., 120) can also be used. A catheter or series of catheters (e.g., 101) are advanced into the heart and are configured to make recordings in and even pace the heart. The catheters may, for example, be advanced into the right atrium (105), though the interatrial septum to the left atrium (107) and into pulmonary vein 103.

Typically, multiple catheters are placed simultaneously. On occasions, due to limited access and inability to simultaneously record and measure using the ablation catheter, the same access site has to be shared by the recording and/or measurement catheter and the ablation catheter. In this case, a recording and/or measurement catheter is inserted, recordings and/or measurements are made, and then the recording and/or measurement catheter is withdrawn. An ablation catheter (101) is then advanced into the heart and positioned at a predetermined site. A number of burns are then performed. The process is performed iteratively as necessary. It is not uncommon for ablation catheters to have to be removed and cleaned before being reintroduced to complete subsequent burn(s). Often, blood clots form on the electrodes of an RF catheter, making it ineffective. Also, catheters of differing configurations may have to be used.

Only one catheter is depicted in FIG. 1, but multiple catheters may be used simultaneously. For instance, a conventional pulmonary vein isolation procedure that uses multiple catheters simultaneously requires four components: a first positioning/anchoring sheath; a recording/measurement/positioning catheter which is inserted through the first sheath; a second positioning/anchoring sheath; and an ablation catheter which is inserted through the second positioning/anchoring sheath. While the simultaneous use of these catheters avoids the need to repeatedly withdraw and insert the catheters, the use of multiple catheters is much more invasive than the use of a single catheter at a time (or a single luminal catheter with a single ablation catheter inserted through the luminal catheter that functions both as a sheath and a recording/measurement/positioning/anchoring catheter).

In conventional cardiac ablation procedures, gaps in an ablation lesion can make the ablation ineffective and can possibly be arrhythmogenic (possibly creating a circuit around the lesion). A labyrinth-like arrangement of conductive tissue resulting from an ablation can effectively create a circuit with a delay which is sufficient to reinitiate a wave of activation after the refractory period of the local cells, but before it would be initiated by proper pacing.

FIGS. 2A and 2B are diagrams illustrating the creation of lesions in cardiac tissue using conventional ablation techniques. FIG. 2A shows a cutaway view of a heart. On the left side of the figure, a single-element contact ablation catheter (211) is shown. On the right side of the figure, a multiple-element contact ablation catheter (221) is shown. (It should be noted that the two different types of ablation catheters may or may not be used at the same time.)

Single-element ablation catheter 211 must be repositioned in a series of adjacent sites so that a series of lesions can be made. The purpose of creating the series of lesions is to overlap the lesions so that a longer, linear lesion is produced. The linear lesion is intended to transect an undesired electrical pathway and thereby disrupt abnormal activation of the cardiac cells. As shown in the figure, incorrect positioning of catheter 211 can result in corresponding mis-positioning of the lesions. For example, lesion 212 is not aligned with the remainder of the lesions 210 created by catheter 211, so there is a gap 213 between the lesions.

FIG. 2B illustrates the propagation of an activation front up to and through lesions 210. The activation front is indicated by the lines (e.g., 214) in the figure. In the case of lesions 210, a remapping of the activation after the ablation would likely show the need for an additional burn to close gap 213. Thus, the misplacement of the catheter results in unnecessary damage to the tissue and adds time to the procedure. Another difficulty may arise because of temporary swelling around the lesions—it is possible that the gap would not be immediately evident. This may add more time to the procedure (in the best case) or possibly require an additional procedure to correct the problem if it is not found during the initial procedure.

Referring again to FIG. 2A, procedures using multiple-element contact ablation catheter 221 may also experience problems in creating the desired lesions. For instance, because the ablation units on the catheter are positioned along the length of the catheter, it may be difficult to position the catheter to create a lesion that is transverse to the direction from which the catheter is inserted (i.e., into the page in the figure). Also, it may be difficult to ensure that the entire length of the catheter along which the ablation units are positioned is in contact with the tissue. As depicted in FIG. 2A, ablation unit 222 of catheter 221 is not in contact with the tissue, so the element cannot reliably deliver the intended dosage of energy to ablate the tissue. As a result, lesions 220 may not serve the intended purpose.

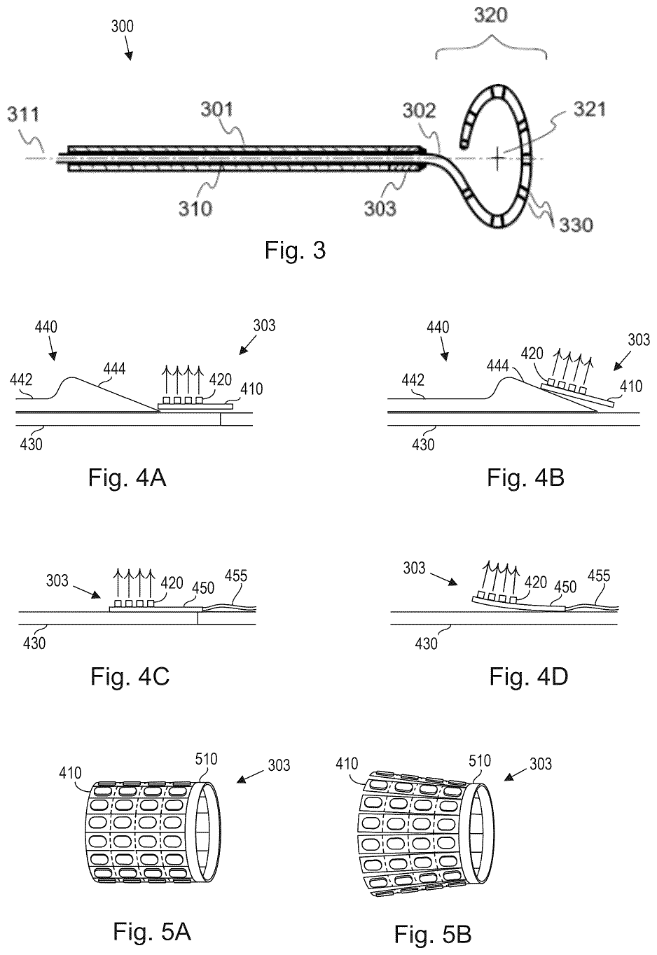

FIG. 3 is a diagram illustrating an exemplary ablation catheter system 300 in accordance with some embodiments of the invention. This figure depicts an ablation luminal catheter 301 which is used in conjunction with a positioning catheter 302. Both ablation catheter 301 and positioning catheter 302 may also serve other purposes, such as providing recording or measurement functions, anchoring the catheters, etc. Ablation catheter 301 includes a directional non-contact ablation unit 303 which is located at or close to the tip of the catheter. Non-contact ablation unit 303 may, for example, be an ultrasound transducer which is configured to deliver energy in the form of ultrasonic waves to the tissue targeted for ablation. The transducer elements of the unit are movable to alter the direction in which the ultrasonic waves are propagated. The ultrasound energy destroys the tissue by heating the tissue, creating lesions that can block unwanted electrical pathways. The ultrasound energy is delivered from a stand-off position, rather than having the ablation unit in contact with the target tissue. The ultrasonic waves can travel through fluids between the ablation unit and the tissue so that the target tissue, rather than the intervening fluid, is destroyed. As noted above, recording elements can also be mounted on both the luminal catheter and the positioning catheter in order to provide recordings and measurements before, during, and after the ablation of the target tissue.

Ablation catheter 301 is itself a luminal catheter. Positioning catheter 302 can therefore be inserted within the lumen of ablation catheter 301, much like using a sheath. Conventionally, a sheath in an ablation procedure serves no purpose other than to provide a conduit through which a functional (e.g., ablation or recording/measurement) catheter is inserted. By incorporating an ablation unit and possibly several recording elements onto the luminal catheter/sheath (or alternatively incorporating a lumen into an ablation catheter), twice as many functional instruments can be inserted into the body with no increase in the invasiveness of the procedure, and no increase in the trauma to the affected tissue which is caused by the insertion procedures. This provides a substantial advantage over conventional techniques. With regard to procedures in which separate ablation and recording/measurement catheters are repeatedly inserted and withdrawn from the body, this embodiment reduces the amount of time required to perform the procedure and reduces the possibility of catheter positioning errors. With respect to procedures in which multiple catheters are simultaneously inserted into the body, this embodiment reduces the amount of space occupied by the surgical instruments because it reduces the number of catheters and sheaths, from typically four (two sheaths, an ablation catheter and a recording/measurement catheter) to two (a luminal catheter/sheath for ablation and recording that also serves as the conduit for a second catheter that performs recording, positioning and/or anchoring functions). The presently disclosed catheter system thereby reduces the trauma to the body.

In the embodiment of FIG. 3, positioning catheter 302 has a main body 310 and a distal portion 320, which includes an array of electrodes (e.g., 330). The distal portion 320 can be formed into a loop. The loop is placed in contact with the tissue (e.g., the myocardium or blood vessel wall) and enables the accurate positioning and stable anchoring of ablation catheter 301 and specifically ablation unit 303, which can be moved forward or backward over main body 310. In this embodiment, loop portion 320 is substantially concentric with and perpendicular to the axis of main body 310 of catheter 302. As a result, if loop center 321 is coaxial with a cavity in which the catheter is inserted, ablation unit 303 remains substantially centered in the cavity, regardless of the movement of ablation catheter 301 over main body 310.

In an alternative embodiment, the main body of the recording/positioning catheter is off-axis rather than being concentric with the loop portion. That is, the axis of main body does not pass through the center of the loop portion. This embodiment may be useful because, when the loop portion is positioned against the walls of a cavity, movement of the ablation catheter over the main body changes the position of the ablation unit with respect to the axis of the cavity. In other words, when the ablation catheter is moved forward (toward the loop), the ablation unit moves closer to the wall of the cavity, and when the ablation catheter is moved backward (away from the loop), the ablation unit moves farther from the wall of the cavity. By changing the distance between the ablation unit and the wall of the cavity (which contains the target tissue), the dosage of energy delivered to the tissue and the area of the target tissue can be adjusted.

Another alternative embodiment may use a positioning/anchoring catheter that does not have a loop at the distal end of the catheter, but instead has a hook-shaped portion. This hook-shaped portion serves essentially the same purpose as the loop portion of the other embodiments in that it is placed against some part of the tissue to stabilize the positioning catheter and allow the ablation catheter to be positioned and anchored by sliding it over the positioning catheter.

The loop shape can be an integral (fixed) feature of the catheter or (re)configurable. The catheter may have a lumen and the distal portion of the catheter may be constructed of a flexible material that takes the shape of a wire that is introduced into the lumen. Alternatively, the shape may be manipulated, as in a deflectable/steerable catheter. In some embodiments, the luminal catheter/sheath may be deflectable/steerable as well.

Another purpose of the loop portions and hook portions of the positioning/anchoring catheters is to enable recording and measurements of the tissue characteristics (e.g., electrical potentials). The positioning/anchoring catheters therefore include electrodes positioned on the loop or hook shaped portions. The electrodes are coupled to recording/measurement/stimulation unit(s) at the proximal end of the catheter. The recording/measurement/stimulation units are configured to transmit stimulus signals to the electrodes if necessary and to receive signals from the electrodes via wiring through the catheter. When the positioning/anchoring catheters are positioned with the respective loop/hook portions against the tissue, the electrodes can be used to record and measure the tissue characteristics. The electrodes may or may not be in contact with the tissue, depending upon the circumstances. Because the positioning/anchoring catheters and corresponding electrodes can remain in place during the ablation procedure, consistent before-and-after recordings and measurements can be made. The electrodes are used in the assessment/evaluation of the effectiveness of an ablation. The electrodes can be positioned to record electrical (cardiac) signals or to stimulate (pace) the heart.

It should be noted that additional electrodes can also be positioned on the ablation luminal catheter/sheath. These electrodes may, for example, be placed on the body of the ablation catheter on the side of the ablation unit opposite the distal end of the catheter. The electrodes of the ablation catheter and positioning/anchoring catheter or sheath would therefore be on opposite sides of the lesion created by the ablation procedure. This allows the operator to assess the effect of the ablation (i.e. whether there is a disconnection or disruption of electrical conduction between the distal and the proximal portion of the tissue) without the need to replace the ablation ensemble with the recording ensemble for this purpose, thus shortening procedure time.

Directional non-contact ablation unit 303 has multiple, individual ablation elements, and provides the ability to change the positions of the ablation elements in order to alter the direction in which the ablation energy is propagated. Referring to FIGS. 4A and 4B, a pair of diagrams illustrating the principle of operation of the directional non-contact ablation unit are shown. In these figures, a partial cross-section of the luminal ablation catheter is depicted. Ablation unit 303 is generally cylindrical and is positioned coaxially around a lumen tube 430 of the luminal catheter. Ablation unit 303 has multiple movable supports 410 on which individual ablation transducer elements 420 are mounted. As depicted in these figures, ablation elements 420 radiate energy in a direction that is substantially perpendicular to the face of movable support 410. The individual elements may radiate energy narrowly in a particular direction, or more broadly. In some embodiments, interference between the energy radiated by different elements may focus the energy or cause the energy to be radiated in a particular pattern.

Ablation catheter 301 has a deformer 440 which is coupled to (i.e., interacts with) the ablation unit. In this embodiment, deformer 440 is positioned coaxially with lumen tube 430 of the catheter and ablation unit 303. Deformer 440 has a proximal portion 442 and a ramp or wedge 444 at its distal end.

Deformer 440 slides axially on the outer surface of lumen tube 430. The proximal end of deformer 440 is coupled to a control mechanism that can cause it to move in one direction or the other.

Deformer 440 is configured to slide from a first position shown in FIG. 4A to a second position shown in FIG. 4B. In the first position, ramp 444 is located adjacent to movable support 410 of ablation unit 303. In this position, deformer 440 does not displace support 410, so the support remains substantially parallel to the axis of the catheter (horizontal in the figure). As deformer 440 is moved distally (to the right in the figures), ramp 444 slides between support 410 and lumen tube 430 of the luminal catheter. Ramp 444 thereby causes the proximal end of support 410 to be pushed away from the axis of the catheter (upward in the figure), tilting support 410 and causing the energy radiated by the transducer elements to be redirected (in this case toward the distal end of the catheter (to the right in the figures).

The deformer is configured to move the supports, and consequently the individual ablation transducer elements, to at least two different positions. In FIG. 4A, the positioning of the supports and ablation elements causes energy to be radiated generally perpendicular to the axis of the catheter, while the positioning of the supports and ablation elements in FIG. 4B causes the energy to be radiated in a direction that is angled forward, toward the distal side of the ablation unit. As can be seen in the figures, the mechanism employed by the deformer can cause the supports and ablation elements to move through a range of positions as the deformer is moved forward (distally) or backward (proximally). In other embodiments, a more discrete mechanism may be used, so that the ablation elements are moved to a set of specific, known positions.

It should be noted that FIGS. 4A and 4B are presented to show the operation of the directional ablation unit in some embodiments at a very high level. Some embodiments may use alternative designs which differ from these figures in various ways. For example, while ramp 444 is depicted as having a linear cross-section (forming a surface having a conic section) which moves the proximal end of support 410 by an amount proportional to the axial movement of deformer 440, the ramp may instead be curved so that the movement of the support is not linear with the movement of deformer. Similarly, while support 410 is depicted as being rigid (so that the support remains flat when moved by the ramp), other designs may use a flexible support which allows the support to flex and curve so that the radiated energy is focused (or defocused) rather than simply being redirected. Various other changes may also be apparent to those of skill in the art upon reading the present disclosure.

Referring to FIGS. 4C and 4D, another alternative design is shown for a movable transducer support mechanism. In this embodiment, rather than using a movable deformer to change the position of the transducer support, an electronic mechanism is used. The transducer support 450 is formed with a shape-memory material that assumes a first shape at a first temperature and assumes a different shape at a different temperature. By controlling the temperature of the transducer support, the shape of the support can be controlled.

For instance, in this embodiment, electrical conductors 455 are connected to support 450 to allow electrical current to be passed through support 450 to control its temperature. An electrical controller (not shown in the figure) is connected to electrical conductors 455 to generate the current through the conductors. When no electrical current passes through the support, the support moves toward (or remains near) the patient's body temperature. As a result, the support assumes (takes on) a corresponding shape, which in this embodiment is flat against the lumen tube 430 as shown in FIG. 4C. With support 450 in this position, transducer elements 420 are positioned in a flat arrangement which radiates energy outward, substantially perpendicular to the surface of lumen tube 430. When current passes through the support, heat is generated, raising the temperature of the support and causing it to take on a different shape (e.g., curved away from lumen tube 430 as shown in FIG. 4D). In this position, transducer elements 420 are positioned in a curved arrangement which radiates energy outward, but slightly to the right in the figure. The curved arrangement also converges or focuses the radiation to some degree. It should be noted that transducer support 450 may be configured to take on any suitable shape, which may be substantially more complex than the simple shapes illustrated in the figure.

Referring to FIGS. 5A and 5B, an exemplary directional non-contact ablation unit is shown. FIG. 5A shows the ablation unit in an undeformed or contracted state, while FIG. 5B shows the ablation unit in a deformed or expanded state. Directional ablation unit 303 comprises a set of elongated supports 410, each of which is connected to a band 510 to form a structure that is generally cylindrical in shape when it is in the undeformed state (as in FIG. 5A). Each support 410 has a corresponding series of ablation transducer elements 420 which are mounted on the support. In the embodiment of FIGS. 5A and 5B, there are 16 supports, and each support has four transducer elements that are evenly spaced along the length of the support. Supports 410 are only connected to band 510, and are not connected to each other. The supports are allowed to flex or pivot at the connection to band 510, so that the supports can tilt outward, away from the axis of the lumen catheter. Consequently, when the ramp portion of the deformer is positioned between supports 410 and the lumen tube 430 of the luminal catheter, the supports are pushed outward by the ramp. This causes the structure to have the general shape of a conic section.

Directional ablation unit 303 may be formed in a number of different ways. In one embodiment, the ablation transducer elements 420 may be formed as a two-dimensional array of elements on a flat substrate. Electrical conductors (e.g., wires and/or electrical traces) can be formed on the substrate for connection to a control cable of the ablation catheter. The substrate can then be cut to separate columns of the array, leaving a strip along one end of the substrate that remains connected to each column (see FIG. 10, where the dashed lines represent the cuts). The columns will become the supports 410 of the ablation unit. The substrate is then wrapped into a cylindrical shape and the ends of the uncut strip are connected to each other to maintain the cylindrical shape.

It should be noted that the structure of FIGS. 5A and 5B is exemplary, and alternative structures may be used in some embodiments. For example, rather than using rigid support structures that flex at a connecting band, the transducer elements may be mounted on a more flexible structure, such as an elastomeric sleeve which can be positioned over the lumen tube of the luminal catheter. This elastomeric sleeve would be designed to stretch and conform to a movable surface which is positioned between the sleeve and the lumen tube of the catheter, such as the deformer described above.

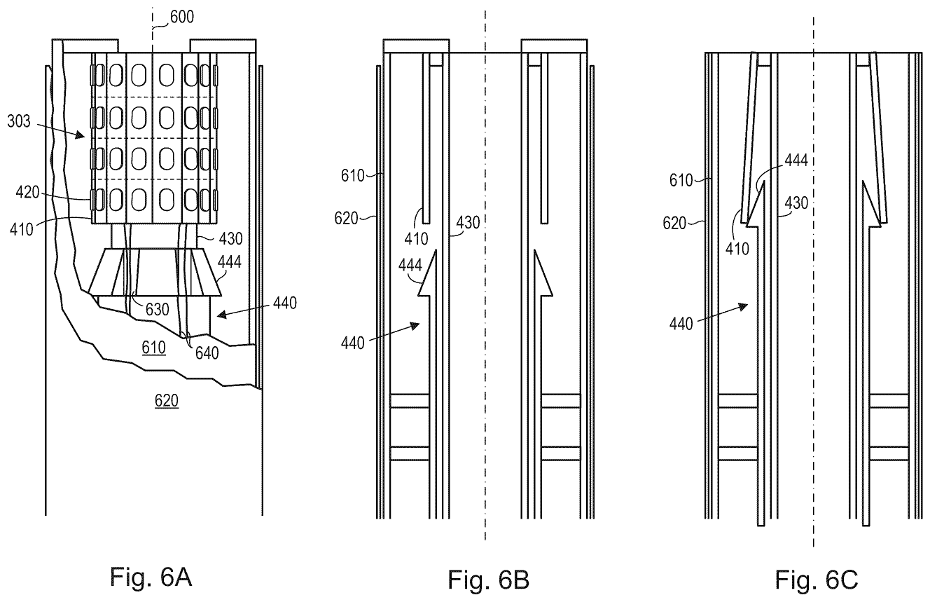

As noted above, the directional ablation unit is positioned coaxially around the lumen tube of the luminal catheter. This is shown in FIG. 6A, which is a partially cut-away view of a portion of an ablation catheter including the directional non-contact ablation unit of FIGS. 5A and 5B. As depicted in this figure, ablation unit 303 is installed over a lumen tube 430. Ablation unit 303 encircles lumen tube 430 and is coaxial with the lumen tube. Lumen tube 430, ablation unit 303 and deformer 440 are positioned coaxially within a pair of outer tubes 610 and 620. There is a gap between ablation unit 303 and lumen tube 430 (as can be seen in FIGS. 6B and 6C) so that a ramp portion 444 of deformer 440 can be inserted between the ablation unit and the lumen tube to force the supports of the ablation unit radially outward, away from the axis 600 of the catheter.

In this embodiment, the ramp portion 444 of deformer 440 is segmented into sections with spaces 630 between them. The spaces are provided to allow wires 640 to extend from the fixed end of ablation unit 303, past ramp portion 444 to the proximal end of the catheter so that they can be connected to an electrical controller that supplies electrical signals to the individual ablation elements. Since wires 640 are positioned in spaces 630, ramp portion 444 can be advanced between lumen tube 430 and ablation unit 303 to deform the ablation unit without being constrained by the wires, and without damaging the wires or their connections to the ablation elements. Deformer 440 may be moved axially in one embodiment using a spiral mechanism at the proximal end of the catheter which allows a user to rotate a handle or dial of the mechanism to advance or retract the deformer to a desired position (thereby moving the ablation elements to desired positions).

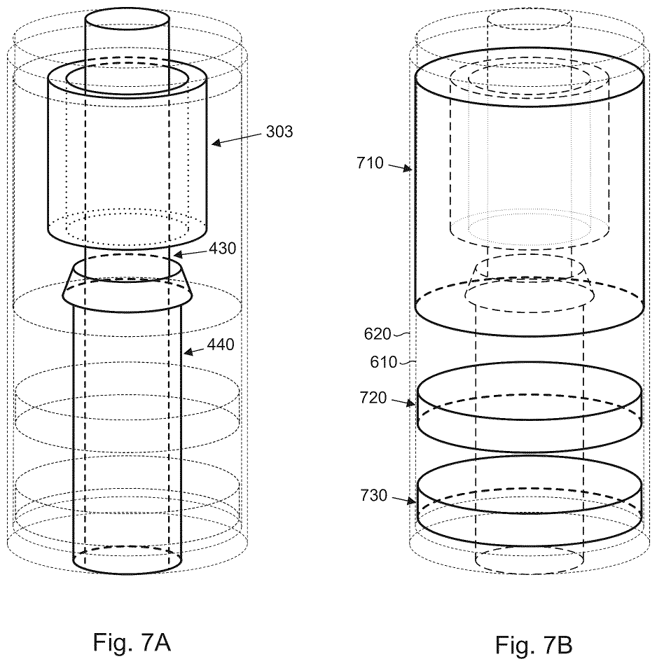

Referring to FIGS. 7A and 7B, another set of diagrams illustrating subsets of the catheter components are shown. In each of these figures, several components are highlighted to more clearly show their relationships to each other. Referring to FIG. 7A, ablation unit 303, deformer 440 and lumen tube 430 are shown using solid lines. Again, it can be seen that ablation unit 303 and deformer 440 are coaxially positioned around lumen tube 430. The position of ablation unit 303 is fixed axially with respect to lumen tube 430 by an adhesive or other means. Deformer 440, on the other hand, is movable axially with respect to lumen tube 430 (vertically in the figure). Outer tubes 610 and 620 are shown in dotted lines in this figure.

Referring to FIG. 7B, a perfusion chamber 710 and two electrodes (720, 730) are depicted using solid lines, while the ablation unit 303, deformer 440 and lumen tube 430 are shown using dotted lines. Outer tubes 610 and 620 are also depicted using dotted lines in this figure.

Perfusion chamber 710 surrounds ablation unit 303. Lumens within tube 610 allow a fluid such as saline to be circulated into perfusion chamber 710 for the purpose of cooling the ablation elements. Tube 610 may also include means such as memory wires to steer the catheter. Tube 620 may be provided as a movable cover for the ablation unit and perfusion chamber. Tube 620 may be advanced to cover the ablation unit and perfusion chamber while the catheter is being advanced into the body, and may be retracted to uncover the ablation unit and perfusion chamber when the catheter has been positioned and is ready for use.

Electrodes 720 and 730 are positioned in the catheter on the proximal side of ablation unit 303. These electrodes may be employed during use of the catheter to take measurements of the electrical characteristics associated with the tissue surrounding the catheter. Electrodes 720 and 730 may also be used in conjunction with electrodes that are located on the distal side of ablation unit 303 on a positioning catheter to take measurements across target tissue which is affected by the ablation energy radiated from ablation unit 303.

Referring again to FIGS. 6B and 6C, cross-sectional views of the catheter are shown to illustrate the operation of the mechanism used to deform/deflect the support structures of ablation unit 303 using deformer 440. In FIG. 6B, deformer 440 is retracted so that it is not in contact with the supports 410 of ablation unit 303. Supports 410 are therefore in their undeformed or contracted position (which might also be considered a relaxed or rest state of the ablation unit). With the supports in this position, the corresponding ablation elements radiate energy in corresponding directions (generally radially outward, away from the axis of the catheter. In FIG. 6C, deformer 440 has been advanced into the gap between supports 410 and lumen tube 430, forcing the lower end of each support radially outward, away from the catheter axis. Consequently, the supports are angled forward so that the energy radiated by the individual ablation elements on each support is redirected in a more distal (upward in the figure) direction.

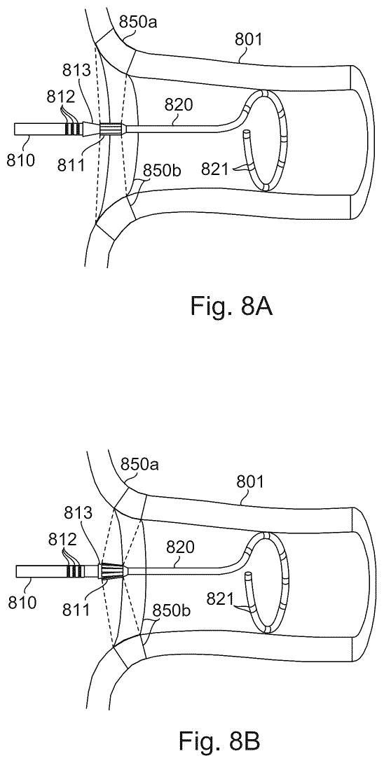

FIGS. 8A and 8B are diagrams illustrating one embodiment of the present system positioned for an ablation procedure in a pulmonary vein 801. Directional non-contact ablation unit 811 is mounted at the distal end of a sheath-like ablation catheter 810. The ablation catheter has a lumen and is configured with positioning catheter 820 in the lumen. Positioning catheter 820 is configured in this embodiment to serve recording and measurement functions as well as positioning and anchoring the ablation catheter. An array of electrodes (821) is mounted on positioning catheter 820. These electrodes are positioned in contact with the walls of the pulmonary vein near the entrance to the left atrium. Reference electrodes 812 are mounted on the body of the ablation catheter. Recordings and measurements from some combination of electrodes may be made before, during, and after an application of energy (e.g., ultrasound energy) for ablation. The recordings and measurements may be used to make assessments of the disease state and of the treatment effect.

Directional non-contact ablation unit 811 is configured in this embodiment to emit energy (e.g., ultrasound energy) in a circumferential pattern and to thereby produce a circumferential burn that results in a continuous lesion such as 850. Lesion 850 isolates the tissue of the pulmonary vein from the cardiac tissue. In an alternative embodiment, the transducer could be configured to generate a partial circumferential burn, or a shorter linear burn. The shape and extent of the burn and the characteristics of the resulting lesion will depend upon various factors, such as the characteristics and positioning of the ablation catheter, and the particular signals that are applied to the individual ablation elements of the ablation unit by the catheter controller.

The characteristics of the burn and resulting lesion will also depend upon the positioning of the ablation elements resulting from the interaction of the deformer and the support structures on which the ablation elements are mounted. In FIG. 8A, deformer 813 is in the retracted position so that it does not cause deformation of the generally cylindrical configuration of the ablation elements on ablation unit 811. Accordingly, the energy emitted by the ablation unit is radiated in a direction that is generally perpendicular to the axis to the ablation catheter (at the ablation unit), forming a lesion 850 which is even with the position of the ablation unit. In FIG. 8B, the deformer 813 has been advanced to a position between ablation unit 811 and the underlying lumen tube, so that the ablation unit is deformed to a conic section configuration. This causes the energy emitted by the ablation unit to be radiated in a direction that is angled distally (forward, toward the distal end of the catheter to the right side of the figure), forming a lesion 850 which is forward from with the position of the ablation unit (to the right of the ablation unit in the figure). It should be noted that the amount by which the emitted energy is angled forward depends on the specific configuration of the deformer and ablation unit and the positioning of the deformer. In some cases, the deformer and ablation unit may be configured to angle the emitted energy proximally (rearward, toward the left side of the figure).

A procedure using the present system could take different forms. The ablation catheter/sheath could be introduced first and the recording catheter then advanced through the ablation catheter/sheath. The recording catheter could be introduced first and the ablation catheter/sheath then advanced over the recording catheter. A guide wire could be positioned first with either or both of the ablation and recording catheters then advanced.

In one embodiment, the recording/positioning catheter is introduced and positioned within the heart. Some number of recordings/measurements is made and an ablation site (or sites) is determined. An ablation catheter/sheath such as one described above is advanced over the positioning catheter to the required position in the heart. Baseline measurements are made with the ablation catheter in place. Then, ablation is performed while recording additional measurements. When it appears that a sufficiently large lesion has been created, the ablation is stopped. With the ablation system still in place, measurements continue and the burn is assessed. If needed, additional burns are made and the ablation procedure is concluded. Note that the ablation catheter/sheath need not be removed to allow for measurements to be made, which results in a more efficient procedure. Also, with an ablation unit such as the ultrasonic transducer, the formation of blood clots or charring on the device is not as likely as with conventional radio frequency ablation systems, so fewer or no cleanings are necessary.

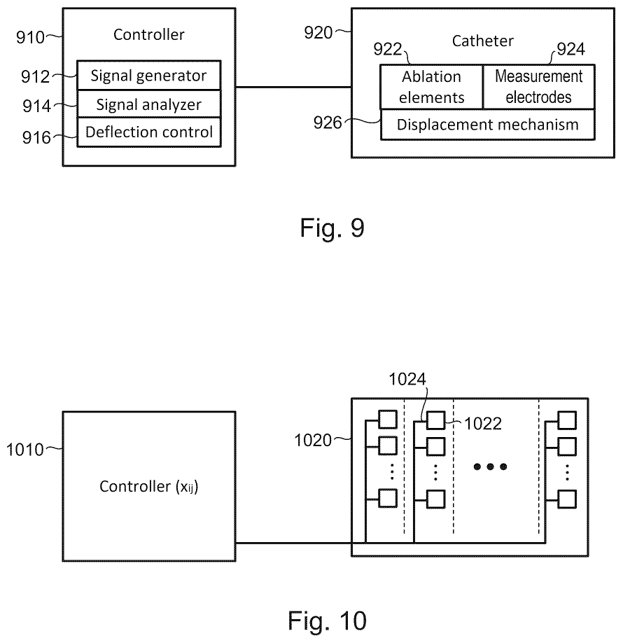

In some embodiments, the system described above is used in conjunction with an electronic controller that is coupled to the catheter(s). The controller may be coupled to the catheters as depicted in FIG. 9 so that it may control the operation of the various electronic components. Controller 910 may, for example, include such components as a signal generator 912, signal analyzer 914, and deflection control system 916. Signal generator 912 may generate signals that are provided to the individual ablation elements 922 of the ablation unit of catheter 920 in order to cause them to generate the ablation energy that is radiated from the unit. Signal analyzer 914 may be connected to the measurement electrodes 924 in the ablation and positioning catheters in order to receive and analyze signals from these components. Signal analyzer 914 may also be connected to receive signals from ablation elements 922, which may be used as sensing devices. Deflection controller 916 is coupled to displacement mechanism 926 and is configured to cause movement of this mechanism, which in turn causes deflection of the ablation unit support structure and positioning corresponding of the ablation elements.

As noted above, the controller and ablation elements may be configured to enable each of the individual ablation elements to be independently controlled. In other words, a different signal may be provided to each of the ablation elements, causing them to generate ablation energy in a particular manner (e.g., emitting energy at specific power levels, frequencies, etc.) The controller may, of course, also control the ablation elements using identical signals, if desired.

FIG. 10 is a diagram illustrating the independent controllability of the individual ablation elements. As depicted in this figure, ablation unit 1020 has an array of individual ablation elements 1022. For example, the array may have N rows and M columns of ablation elements. In the case of the cylindrical ablation unit described in the examples above, the array may be wrapped around the lumen tube to form a cylindrical unit, in which case there would be N rings, each having M ablation elements (where each of the elongated supports in the ablation unit has one element from each of these rings mounted on it). Controller 1010 is configured to individually address each of these ablation elements via corresponding electrical lines 1024. (For the sake of simplicity, the lines are not separately depicted in the figure.) The controller may thereby adjust the pattern and intensity of the radiated ablation energy and consequently control the burn and resulting tissue lesions resulting from the radiated energy.

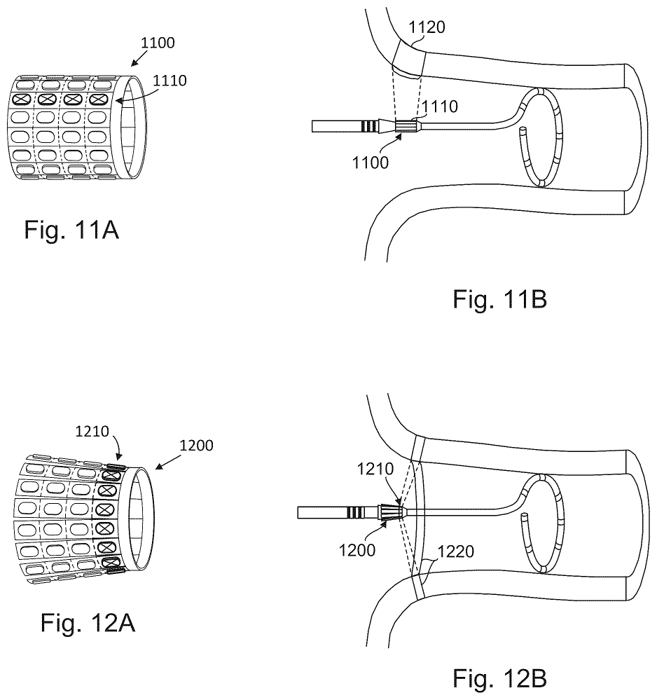

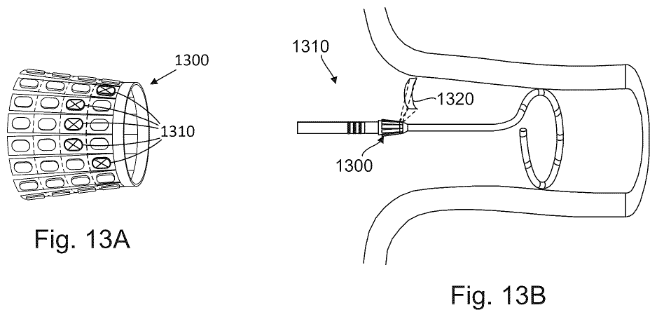

Examples of the ways in which the individual ablation elements can be controlled are shown in FIGS. 11-13. FIGS. 11A and 11B show a single column 1110 of ablation elements in ablation unit 1100 which are activated to create a linear lesion 1120 on a vein, where the lesion is substantially parallel with the axis of the ablation catheter. The activated ablation elements are indicated by an “X” on the elements. FIGS. 12A and 12B show a single row 1210 of ablation elements in ablation unit 1200 which are activated to create a circumferential lesion 1220 on the vein. FIGS. 13A and 13B show a set 1310 of ablation elements in ablation unit 1300 which are activated to create a curved lesion 1320 on the vein. The ablation elements can be individually turned on and off by the controller to generate any desired pattern. The controller may also vary the levels of energy emitted by the different ablation elements (e.g., a first element emits full power while another element emits half power) in order to further control the pattern of ablation energy emitted by the ablation unit, and thereby control the generated lesion.

There may be numerous alternative embodiments of the system. It is contemplated that the invention is not limited to ultrasound ablation, and various different non-contact ablation modalities can be used. The selection of materials from which a catheter body and other components can be constructed is also quite varied and may include such materials as polyurethane, silicone, PTFE, steel, copper and carbon. The systems described above comprise an ablation catheter/sheath with a lumen within which the recording/positioning catheter is positioned. The system can alternatively comprise a recording/measurement catheter with a lumen, within which the ablation catheter is positioned. The distal portions of the catheters can be of a variety of shapes (e.g., circular, semi-circular, hook, Y, basket). The catheters may comprise lumens to make use of guide wires for positioning or shaping purposes. A deflection mechanism could be incorporated for purposes such as steering or shaping the catheters. There are many possible arrangements of electrodes for testing, recording, measurement, pacing, reference, etc.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein and recited within the following claims.

What is claimed is:

1. An ablation catheter system comprising:
a catheter, having a lumen tube;
a non-contact ablation unit connected to the catheter, wherein the non-contact ablation unit comprises a plurality of ablation elements and a plurality of supports, wherein each of the plurality of ablation elements emits energy, and wherein each of the plurality of ablation elements is mounted on a corresponding one of the plurality of supports; and
a deformer axially movable along the lumen tube of the catheter between:
a first position in which the deformer is located outside of an annular space defined between the lumen tube and the plurality of supports; and
a second position in which the deformer is located within the annular space, wherein the deformer displaces at least a portion of each of the plurality of supports in a radially outward direction with respect to an axis of the lumen tube thereby changing orientation of each of the plurality of ablation elements to thereby change direction of the energy emitted by each of the plurality of ablation elements.

2. The ablation catheter system of claim 1, wherein the deformer includes a ramp portion.

3. The ablation catheter system of claim 2, wherein the ramp portion contacts the elongated supports when the deformer is in the second position.

4. The ablation catheter system of claim 2, wherein the ramp portion is linear to move the plurality of supports by an amount proportional to an axial movement of the deformer.

5. The ablation catheter system of claim 2, wherein the ramp portion is non-linear to move the plurality of supports by an amount that is nonproportional to an axial movement of the deformer.

6. The ablation catheter system of claim 1, wherein the deformer is positioned coaxially with the lumen tube.

7. The ablation catheter system of claim 1, wherein each elongated support of the plurality of elongated supports is connected at a first end to a fixed band, and wherein the deformer is configured to contact a second end of each of the plurality of elongated supports and thereby displace each elongated support of the plurality of elongated supports and corresponding ablation element.

8. The ablation catheter system of claim 7, wherein the plurality of elongated supports and the fixed band are in a contracted position which has a generally cylindrical shape when the deformer is in the first position.

9. The ablation catheter system of claim 7, wherein the plurality of elongated supports and the fixed band are in an expanded position which has a generally frusto-conical shape when the deformer is in the second position.

10. The ablation catheter system of claim 1 wherein the plurality of ablation elements comprises ultrasound transducers configured to emit ultrasonic energy.

11. The ablation catheter system of claim 1, wherein the catheter is a first catheter, wherein the ablation catheter system further comprises a second catheter configured to fit within the lumen of the first catheter, wherein the second catheter is configured to stabilize the non-contact ablation unit relative to a target tissue.

12. The ablation catheter system of claim 11, wherein the second catheter comprises a loop or hook shaped portion at a distal end to aid in maintaining proper positioning in the ablation catheter system.

13. The ablation catheter system of claim 11, wherein at least one of the first and second catheters includes one or more measurement electrodes configured to measure electrical characteristics of the target tissue.

14. The ablation catheter system of claim 1, wherein the plurality of ablation elements are electrically coupled to a controller, wherein the controller is configured to independently control each of the plurality of ablation elements.

15. The ablation catheter system of claim 14, wherein the plurality of ablation elements is selectively activated, and wherein the selectively activated plurality of ablation elements produces one or more lesions at a target tissue without repositioning the ablation catheter system.

16. The ablation catheter system of claim 15, wherein when the deformer is in the second position, the selective activation of each of the plurality of ablation elements causes the energy emitted by each of the plurality of ablation elements to be focused onto the target tissue or radiated in a particular pattern into the target tissue.

17. A method for ablating tissue using an ablation catheter system, the method comprising:
inserting a first catheter, having a lumen tube, into a patient;
inserting a second catheter into the lumen of the first catheter;
positioning a distal end of the second catheter in contact with a target tissue;
positioning the first catheter, having a non-contact ablation unit, adjacent to the target tissue;
positioning an axially movable deformer into a first position, wherein the deformer is located outside an annular space defined between the lumen tube and a plurality of supports; and
moving the deformer to a second position located within the annular space, wherein the deformer displaces at least a portion of each of the plurality of supports in a radially outward direction with respect to an axis of the lumen tube thereby changing the direction of the energy emitted by each of the plurality of ablation elements.

18. The method of claim 17, wherein the step of moving the deformer to the second position further comprises:
increasing a radial force applied to the plurality of elongated supports as the deformer moves through the annular space.

19. The method of claim 17, the method further comprising:
selectively activating a subset of the plurality of ablation elements to produce one or more lesions at the target tissue.

20. The method of claim 17, wherein the step of moving the deformer further comprises:
aligning a specific cross-sectional profile of the deformer with the plurality of ablation elements to radially expand the elongated supports for localized tissue ablation.
